(19) United States
(12) Patent Application Publication
(10) Pub. No.: US 2016/0278962 A1
(43) Pub. Date: Sep. 29, 2016

(54) Wearable Active Upper Body Support System

(71) Applicant: Frank Xiaoqing Zhang, Fremont, CA (US)

(72) Inventor: Frank Xiaoqing Zhang, Fremont, CA (US)

(21) Appl. No.: 14/821,581

(22) Filed: Aug. 7, 2015

Related U.S. Application Data
(60) Provisional application No. 62/137,816, filed on Mar. 25, 2015.

(51) Int. Cl. A61F 5/02; A61F 2/70

(52) U.S. Cl. CPC A61F 5/026; A61F 5/024; A61F 2/70; A61F 2002/701; A61F 2002/704

(57) ABSTRACT

A wearable active upper body support system having active shoulder support device, battery, and waist belt. The active shoulder support device is comprised of shoulder support actuator(s) and controller. The system provides counter gravity relief to spine by position the shoulder support actuators along sides of the ribcage under the left and right armpits, with actuator mounting bases anchored in mounting pockets on the waist belt, and the shoulder support pads exerting microcontroller controlled active upper bound force from under the shoulders. The system can be concealed under loose clothing and is wearable with no constraint on daily activities such as walking around, slow running, standing, sitting, or lying down.

### RELATED APPLICATIONS

The present application is a continuation-in-part application of U.S. provisional patent application, Ser. No. 62/137,816, filed Mar. 25, 2015, for WEARABLE ACTIVE UPPER BODY SUPPORT SYSTEM, by Frank Xiaoqing Zhang, included by reference herein and for which benefit of the priority date is hereby claimed.

### FIELD OF THE INVENTION

The present invention relates to body support or back support and, more particularly, to an upper body support system which provide computer-controlled, actuator driven, upper bound shoulder support force for stress relief on spine, which is battery powered and wearable, hence, allowing the user the flexibility of sitting, standing, walking, and even slow running, while the system is operating.

### BACKGROUND OF THE INVENTION

Human spine is not ideally structured to handle the gravity force from upper body, resulting in numerous abnormal conditions such as herniated discs, sciatica pain and scoliosis, to name a few, which are common, painful and difficult to cure.

There is a strong need for devices to help spine handle the stress from upper body gravity force. Hundreds of inventions have been filed over the years attempting to address the need. Those inventions and devices allowing user the flexibility and mobility of normal daily life activities such as walking around are all passive devices with limited functionality, and can be divided into three (3) categories. They are back braces, mechanical support, inflatable or pneumatic devices. Back braces are wrapped tightly around the body surrounding the spinal cord. They are commonly used particularly in women to prevent and treat spinal cord from excessive curving under stress. They do allow the flexibility and mobility of the wearer. Mechanical (back, body, or shoulder) support devices are typically fixture mounted on ground, chair, bench, or bed which provide passive support and are somewhat effective in relieving the spine from stress due from the upper body gravity. Current inflatable or pneumatic devices for (back, body, or shoulder) support are similar to the mechanical devices in relieving the spine from stress. However, they are typically bulky and require the user to either lie down or be constrained with the devices in use.

Devices for lumbar traction and spinal decompression therapies provide active force to losing up the stressed spinal joints. During the therapy, patient lies down on specialized bench (or table), with shoulders strapped down or stopped from moving on one end, and waist or lower strapped to a lumbar belt, and the body (spinal cord) between the two strapping areas are being stretched by (may be computer controlled) pulling force applied to the lumbar belt. Lumbar traction and spinal decompression therapies are very popular with patients suffering from spinal cord illness.

Back braces do not relieve spine from the upper body gravity induced stress. Back braces are passive and not very effective in back pain relief, and it is not comfortable to wear. Mechanical support devices are effective in reliving the spine from stress due from the upper body gravity. However, mechanical support devices are passive, not convenient and not comfortable to use. They are either stationary in nature to not allow user mobility or not graceful enough (in the case of crutches) for daily use in public. Current inflatable or pneumatic support devices are comfortable to use. However, they are typically bulky in size, vulnerable to damages and air leakage in particular, and hence not suited for daily, mobile usage.

Lumbar traction and spinal decompression therapies are very popular with patients suffering from spinal cord illness. However, they are not used to relieve spinal cord from upper body gravity induced stress. As patient must lie down and be strapped to bench or table top, their use can not be mobile.

### SUMMARY OF THE INVENTION

In accordance with the present invention, there is provided a wearable active upper body support system having active shoulder support device, battery, and waist belt. The active shoulder support device has shoulder support actuator(s) and controller. The shoulder support actuator has a shoulder support pad mounted on top of a power-driven linear actuator, enhanced with multiple sensors reporting back to the controller. The controller has a microcontroller-based control board taking feedback from the sensors on the actuator, user commands from control panel, configuration from touch-sensitive display, driving electric motor which power the linear actuator. The waist belt (or waist brace) is a flat band with two connecting ends to be tightened around the lumbar to pelvis area, with mounting pockets built around the waist belt for housing actuator, battery, and controller, serving as the anchoring base. Combined, the system provides counter gravity relief to spine by position shoulder support actuators along sides of the ribcage under the left and the right armpits, with actuator mounting bases anchored in mounting pockets on the waist belt, and the shoulder support pads exerting microcontroller controlled active upper bound force from under the shoulders. The wearable active upper body support system can be concealed under loose clothing and is wearable with no constraint on daily activities such as walking around, slow running, standing, sitting, or lying down.

It would be advantageous to provide a shoulder support actuator with a shoulder support pad mounted on top of power-driven linear actuator for providing active shoulder support to counter gravity force from upper body for spine stress relief.

It would further be advantageous to provide, in the above linear actuator, a rotation-to-linear motion conversion linkage to facilitate using rotational motor as the power source.

It would further be advantageous to provide, in the above linear actuator, ballscrew as the rotation-to-linear motion conversion linkage. Ballscrew is highly efficient. The high efficiency conversion is important in achieving desired long operating time from given battery capacity.

It would further be advantageous to provide, in the above linear actuator, a rotational speed reduction mechanism in the power chain upstream to the rotation-to-linear motion conversion linkage so that high speed motor can be used as the power source. With given power rating, motor with higher speed tends to be smaller in size and lower in cost, which is desired for the shoulder support device to be wearable and mobile.

It would further be advantageous to provide, in the above linear actuator, a belt-pulley mechanism as the above mentioned rotational speed reduction mechanism, which in addition to the speed reduction, belt-pulley is relatively low in noise level and easy to manufacture and maintain.

It would be advantageous to provide a microcontroller-based controller that would be able to run sophisticated control algorithm to provide active shoulder support features which can be optimized for user experience.

It would also be advantageous to provide a load sensor feeding back the actual support force by the shoulder support pad from under the shoulder to the controller on a real-time basis so that the level of spine stress relief can be accurately managed.

It would also be advantageous to provide a rotation sensor, feeding back the above linear actuator's shaft rotation status to the controller. Knowing the shaft rotation and position status, with given pitch, the shoulder support pad travel from its lower limit position can be determined.

It would further be advantageous to provide the above mentioned control algorithm with multiple operating states so that behaviors for the above mentioned active shoulder support device can be clearly defined and managed.

It would further be advantageous to provide the above mentioned control algorithm with multiple operating states running in the above controller, including an Active state as the primary operating state in which the shoulder support actuator(s) provide the desired upper bound shoulder support force.

It would further be advantageous to provide the above mentioned control algorithm with multiple operating states running in the above controller, including a Tracking state in which the shoulder support actuator(s) drive the shoulder support pad(s) maintaining contact with the bottom of the shoulder(s) while not providing meaningful support force. Tracking state is important not only as a transitional step before engaging actual shoulder support force (in Active state), but it provides a flexible and handy transition whenever the user need to engage in certain activities (such as bending, turning upper body hard, or active motion) and want to momentarily stop the active shoulder support force, putting the actuator(s) in Tracking state under these scenarios can facilitate a quick and smooth transition back when such activities are over.

It would further be advantageous to provide the above mentioned control algorithm with multiple operating states running in the above controller, including a Ready state in which the shoulder support actuator is idling while the system is powered and alive, waiting for and responding to certain user inputs. Ready state is important as it provide a handy environment for troubleshooting, adjusting, and configuring the system.

It would be advantageous to provide a waist belt (or waist brace), a flat piece with two connecting ends, made of strong fabric and fastened around the lower waist area right above the hips, serving as the anchoring base for the shoulder support actuator(s) so that the integrated system can be fitted under, above, or sandwiched between clothes, making it wearable, while providing counter upper body gravity relief to the spine.

It would further be advantageous to provide dedicated mounting pockets on the above mentioned waist belt for housing the actuator(s), controller, and battery(es). Here, mounting pocket on the waist belt is a generalized term for structures with opening on top and provide constraint on relative motion down-ward and side-ways. As long as the items in their dedicated mounting pockets are sufficiently constrained from down-ward motion and side-way motion, the mounting pockets do not necessarily have to have bottom(s) or side(s).

It would further be advantageous to provide battery (or batteries) as the primary energy source to the above wearable upper body support system so that the user can move around not being constrained by electric cord, making it mobile in addition to the wearable feature.

It would further be advantageous albeit optional to provide the above mentioned controller, integrating microcontroller-based controller board in a case with multiple command buttons and power switch (or button) on its front panel for easy access and a touch-sensitive display on its rear panel for configuration, monitoring and trouble shooting.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of an embodiment of the active shoulder support device, having actuator(s) and controller, and the wearable active upper body support system, having the active shoulder support device, battery, and waist belt; wiring cables are not shown;

FIG. 2 is an application perspective of the embodiment of the wearable active upper body support system when worn by a person;

FIG. 3 is a presentation of the assembly and its section view for one embodiment of the shoulder support actuator;

FIG. 4 is a presentation of the same embodiment of the shoulder support actuator shown in FIG. 3 which reveal internal details by removing the actuator enclosure, the pulley belt, and the pushrod shell;

FIG. 5 is a perspective of the same embodiment for the shoulder support actuator shown in FIG. 3 and FIG. 4, with both actuator enclosure and pulley belt removed, and a detailed view for the rotation sensor and retract position sensor mounting;

FIG. 6 is a presentation for an embodiment of the controller showing its front panel with power switch and multiple control buttons on the top, and its rear panel with a touch-sensitive display on the bottom; a microcontroller-based control board is enclosed in the controller enclosure; wiring cables are not shown;

FIG. 7 is a diagram showing an embodiment of the functional blocks on the microcontroller-based control board and its connections to other components in the system;

FIG. 8 is a diagram showing an embodiment of a state machine to be implemented in the embedded software (firmware);

FIG. 9 is a flowchart showing the 1st page of an embodiment of the control algorithm to be implemented in the embedded software; and

FIG. 10 is a flowchart showing the 2nd page of the same embodiment of the control algorithm as shown in FIG. 9 to be implemented in the embedded software.

For purposes of clarity and brevity, like elements and components will bear the same designations and numbering throughout the Figures.

### DESCRIPTION OF THE PREFERRED EMBODIMENT

Definition of Terms:

In this document, shoulder support is defined as the upper bound support or force injected from the bottom of shoulder in armpit.

Upper body support is the upper bound support or force applied to counter the gravity force on human upper body, and to push (stretch, or straighten) the upper body away from the lower waist or the hip.

Shoulder support can be used for providing upper body support when combined with waist belt 24 (or waist brace) as its anchoring base.

Active support stands for support motion and force that can be varied or controlled by computer (or microcontroller) following certain algorithm, contrasting to passive support which is not computer controlled.

Mounting pocket 25 on the waist belt 24 is a generalized term for a structure with opening on its top and provide constraint on relative motion down-ward and side-ways. As long as the item in the mounting pocket 25 is sufficiently constrained from down-ward motion and side-way motion, the mounting pocket 25 does not necessarily have to have bottom or side(s).

A General Overview of the Invention:

A wearable active upper body support system 16 having active shoulder support device 18, battery 22, and waist belt 24. The active shoulder support device 18 has shoulder support actuator 20 and controller 26. The shoulder support actuator 20 has a shoulder support pad 28 connected on top of a power-driven linear actuator 27, enhanced with multiple sensors reporting back to the controller 26. The controller 26 has a microcontroller-based control board 82 taking feedback from the sensors mounted on the shoulder support actuator 20, user commands from control panel 65, configuration from touch-sensitive display 80, driving electric motor 48 in the shoulder support actuator 20. The waist belt 24 (or waist brace) is a flat band to be tightened around the lumbar to pelvis area with two connecting ends, with mounting pockets 25 built around the waist belt 24 for housing shoulder support actuator 20, battery 22, and controller 26, serving as the anchoring base for shoulder support actuator 20 in particular. The wearable active upper body support system 16 provides counter gravity relief to spine by position shoulder support actuators 20 along sides of the ribcage under the left and the right shoulders, with actuator mounting bases 38 anchored in mounting pockets 25 on waist belt 24, and shoulder support pads 28 exerting microcontroller controlled active upper bound force from under the shoulders. The wearable active upper body support system 16 can be concealed under loose clothing and is wearable with no constraint on daily activities such as walking around, slow running, standing, sitting, or lying down. When combined with power supply, the active shoulder support device 18 provides the functionality of microcontroller managed upper bound shoulder support. Battery 22 as the energy source enhances mobility and portability. Waist belt 24 is a necessary component providing mounting and anchoring base which make the upper body support system wearable and mobile.

Two features outstanding in the present invention. The first feature is an active shoulder support device 18, comprising shoulder support actuator 20, controller 26 for providing microcontroller actively managed shoulder support exerted by shoulder support pad 28 on top of linear actuator 27. Load sensor 62 in the shoulder support actuator 20 feeds back real-time support force to controller 26, so that the controller 26 can accurately manage the shoulder support force, as well as the force-over-time varying pattern as needed. Depending on the selection of the anchoring for the shoulder support actuator 20, the active shoulder support device 18 can be used in stationary, mobile (or portable) and wearable applications.

The second feature outstanding is a wearable active upper body support system 16 that packages active shoulder support device 18, battery 22, and waist belt 24, with the waist belt 24 serving as the mounting and anchoring base and the design intention to make the system portable and wearable that can be worn by the bearer under, sandwiched, or on top of clothing, providing active upper body support with no constraint on daily activities such as walking around, slow running, standing, sitting. The inclusion of rechargeable battery 22 into the system removes the constraint to portability by power cord.

Ballscrew 55 and leadscrew can have the same set of functionality and mechanical interface which matters to this invention, such as the rotation-to-linear motion conversion and the mechanical dimensions. The key difference is in the use of bearing balls in ballscrew 55 vs no bearing balls in leadscrew, which result in difference in friction, longevity and cost. From the inventor's point of view, ballscrew 55 and leadscrew are interchangeable to the concept of this invention. Particular embodiments may favor the use of ballscrew 55 where active support force, efficiency (hence, battery 22 run time), and longevity of the system are considered more important. Other embodiments may favor the use of leadscrew where initial cost is considered more important, static or semi-static support force and reduced system longevity are acceptable compromise. In the description of the preferred embodiment, ballscrew 55 is used except when alternative or other embodiments are discussed. In the drawings, details on the bearing balls and the mechanism to re-circulate the balls are omitted for simplicity.

Details on Structure and Components of Embodiments:

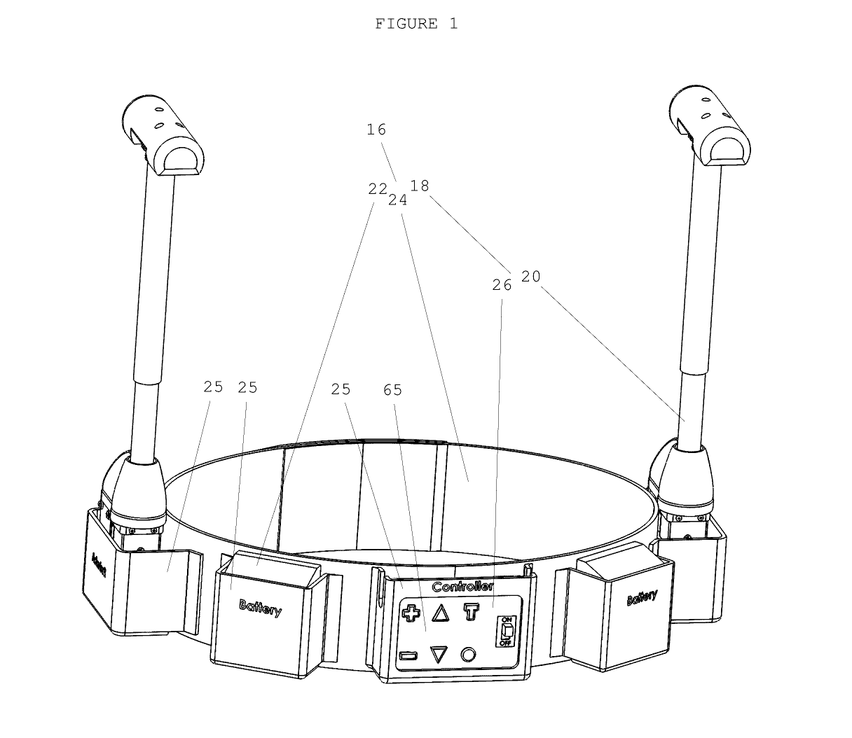

FIG. 1 is a perspective view of one embodiment of the wearable active upper body support system 16, comprised of active shoulder support device 18, battery 22, and waist belt 24. The active shoulder support device 18 has shoulder support actuator 20 and controller 26. Two shoulder support actuators 20 are shown in this embodiment for the two shoulders, left and right. When unbalanced one shoulder support is needed, an embodiment can have only one shoulder support actuator 20. On the other hand, for finer control on shoulder support, more than one shoulder support actuators 20 can be used on each side, which result in other potential embodiments with more than two shoulder support actuators 20. Two rechargeable batteries 22 are shown in this embodiment. In other embodiments, one or more than one batteries 22 can be configured to strike proper compromise among battery 22 capacity, weight and mounting locations according to their particular application requirements. The connecting cables are not shown.

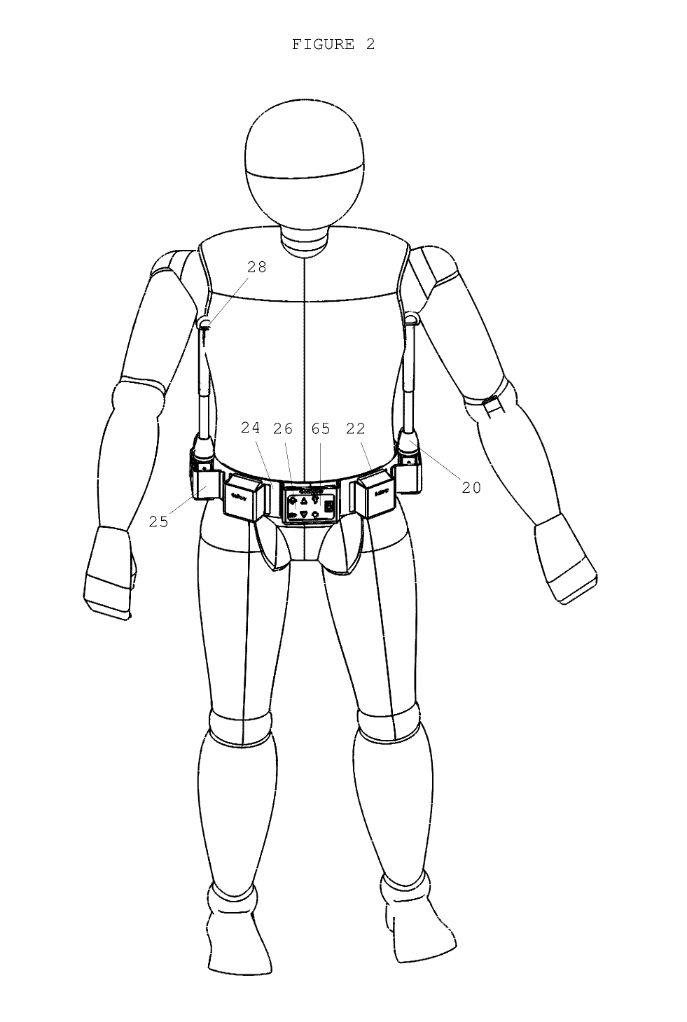

FIG. 2 is an application perspective of the embodiment of the wearable active upper body support system 16 when worn by a person. Two shoulder support actuators 20 are positioned along the two sides of the ribcage under the left and right shoulders, with their shoulder support pads 28 extended into their corresponding armpits reaching the bottom of perspective shoulders, and their mounting bases 38 housed and anchored in their dedicated mounting pockets 25 on the waist belt 24. Two pieces of rechargeable batteries 22 are shown for the embodiment. Controller 26 is housed in its dedicated mounting pocket 25 with its control panel 65 shown through an opening window on the front wall of the mounting pocket 25, and control buttons exposed for easy access. The wiring cables are not shown. The waist belt 24 is shown tightened around the lower waist area right above hips, providing housing and anchoring for the rest of the system.

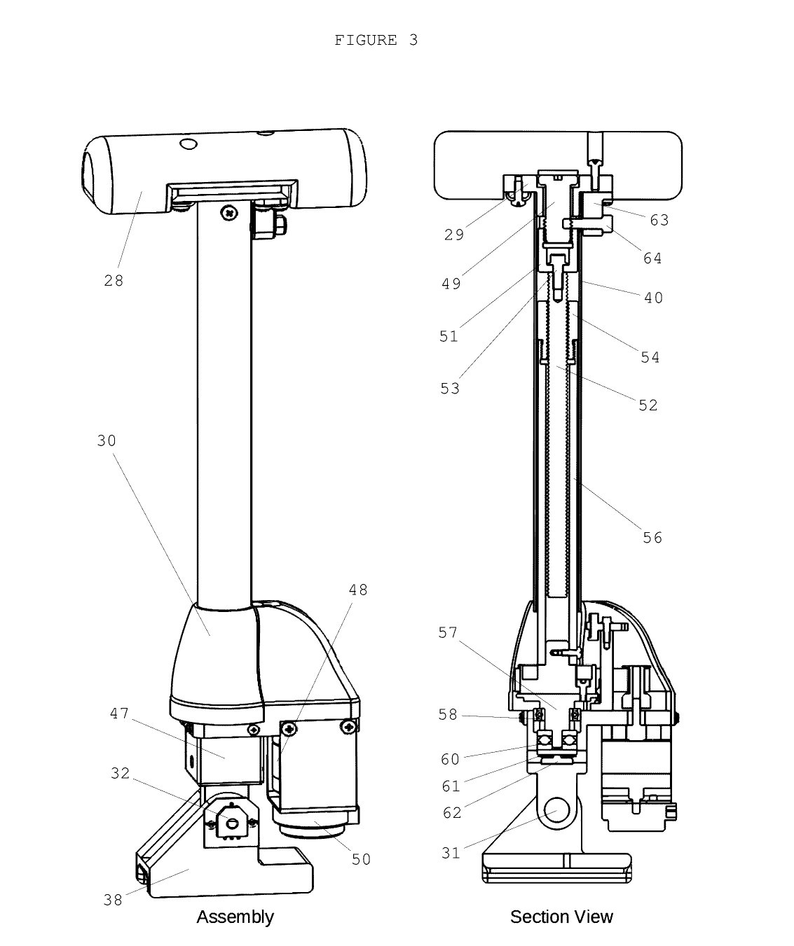

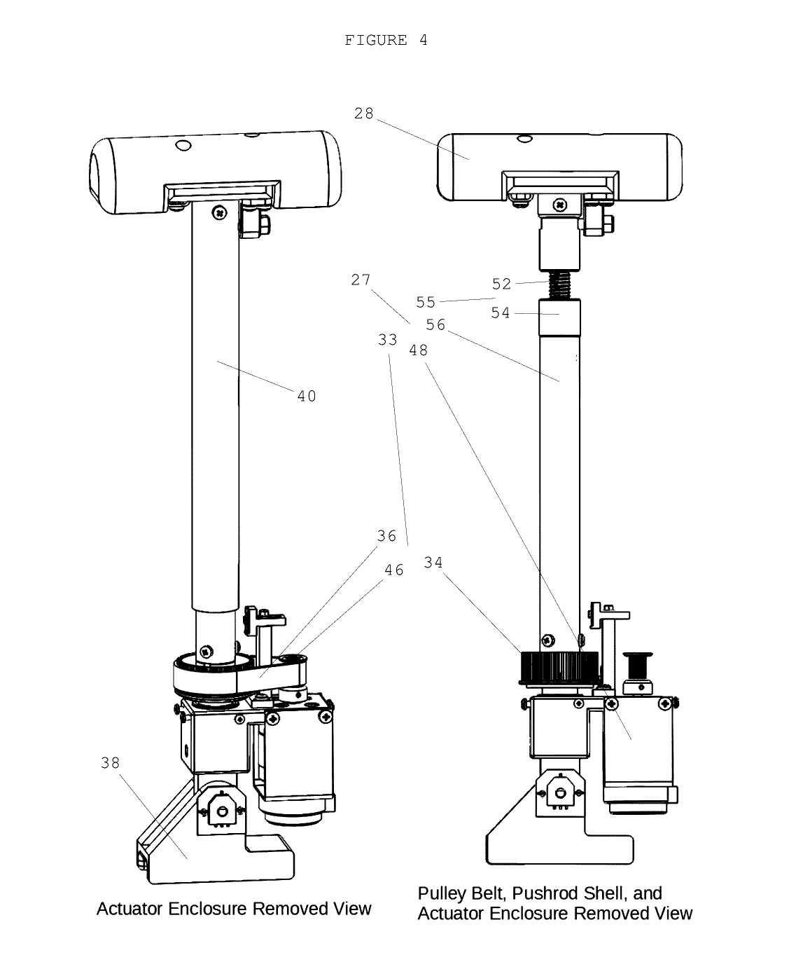

FIG. 3 is a presentation of the shoulder support actuator 20 assembly and its section view. On the top of the shoulder support actuator 20, shoulder support pad 28 has a short beam like top for a comfortable contact when pressed by the shoulder support force to the under part of shoulder. The shoulder support pad 28 is mounted above a power-driven linear actuator (see in FIG. 4). The linear actuator 27 has a rotation-to-linear motion conversion mechanism (a ballscrew 55 set used in this embodiment), driven by the rotational output from a rotation speed reduction mechanism (a belt-pulley 33 set used in this embodiment) covered by the actuator enclosure 30, with electric motor 48 as its power source. On the bottom of the shoulder support actuator 20 is its mounting base 38, with a mounting shaft 31 connecting an actuator housing 47, which has the entire upper part of the shoulder support actuator 20 housed on it, with its mounting base 38. A swing angle sensor 32 is mounted on the mounting base 38 center-aligned with and covering one end of the mounting shaft 31, monitoring and feeding back its swing angle to controller 26 in real-time. For the particular embodiment, rotary position sensor is used for the swing angle sensor 32.

Shown in the section view, a set of journal bearing 58 and thrust bearing 60 are mounted on the support shaft 57 and installed inside the actuator housing 47, supporting the shoulder support pad 28, the associated shafts, and the pulley-driven 34. Under the thrust bearing 60, separated by a thrust end plate 61, is a load sensor 62, which monitor and feed back the actual support force in real-time to the controller 26 for accurate closed-loop control on the support force.

Also shown in the section view is a torque limiter 64. The torque limiter 64 is a pin or plug like device with thread for fastening to torque limiter housing 63. It is the only mechanism limiting the shoulder support pad 28, the pad base 29, the torque limiter housing 63 and the pushrod shell 40 from free rotating against the ballscrew shaft 52, the ballscrew end adaptor 51, the big end holder 49, and the ballscrew end bolt 53. The torque limiter 64 is made of material with known shear property, and shall break off when shear load (which is torque load on shoulder support pad 28) on it goes beyond a pre-determined safety limit.

FIG. 4 is a presentation of the same embodiment shown in FIG. 3 of the shoulder support actuator 20 which reveal the internal details by removing the actuator enclosure 30, the pulley belt 36, and the pushrod shell 40. As shown, in this particular embodiment, a ballscrew 55 set is used to implement the rotation-to-linear motion conversion mechanism. The ballnut 54 in the ballscrew 55 set is fastened to the inner threads on the hollow shaft 56 (see also the section view in FIG. 3) and being driven by the hollow shaft 56. The rotating ballnut 54 back drives the mating ballscrew shaft 52. The rotational motion of the ballnut 54 is translated into up-and-down linear motion by the ballscrew shaft 52. Ballscrew 55 has little friction due to the use of bearing balls, which result in high mechanical efficiency and is ideal for applications where active force-over-time varying pattern in support force is desired as well as if energy efficiency has to be high. On the other hand, if close to static support force (such as stable shoulder support force, slowly step changing shoulder support force, or periodically changing shoulder support force) is desired in other embodiments, leadscrew (or lead screw) set can be used to replace ballscrew 55 set. Leadscrew is simpler in structure and lower in cost than ballscrew 55. Its self-locking feature means no constant push from the power source is required in order to maintain the shoulder support pad 28 position and the shoulder support force. Threadless ball screw is another potential substitute for ballscrew 55 in yet another set of embodiments, as it enjoys the same high mechanical efficiency due to the use of bearing balls in ballscrew 55.

Also in the particular embodiment shown in FIG. 4, a belt-pulley 33 set is used to implement the rotational speed reduction mechanism. In the set, the pulley-driving 46 is mounted and fastened to the motor output shaft; the pulley-driven 34 is concentric and firmly connected to the hollow shaft 56 on its upper end, and the support shaft 57 on its lower end; the pulley belt 36 connects the pulley-driving 46 and the pulley-driven 34, reducing the high input rotation speed on the pulley-driving 46 to the much lower rotation speed on the pulley-driven 34, and augmenting torque in the meantime. Belt-pulley 33 set has relatively lower alignment requirement and lower noise level compare to using gear set for speed reduction. However, if low maintenance is desired, gear set (1 or multiple stages) instead of belt-pulley 33 set for speed reduction will be better fit for such other application embodiments, replacing belt-pulley 33 set. Gear set for speed reduction is not only lower in maintenance, but also higher in mechanical efficiency, compare to belt-pulley 33 set for speed reduction.

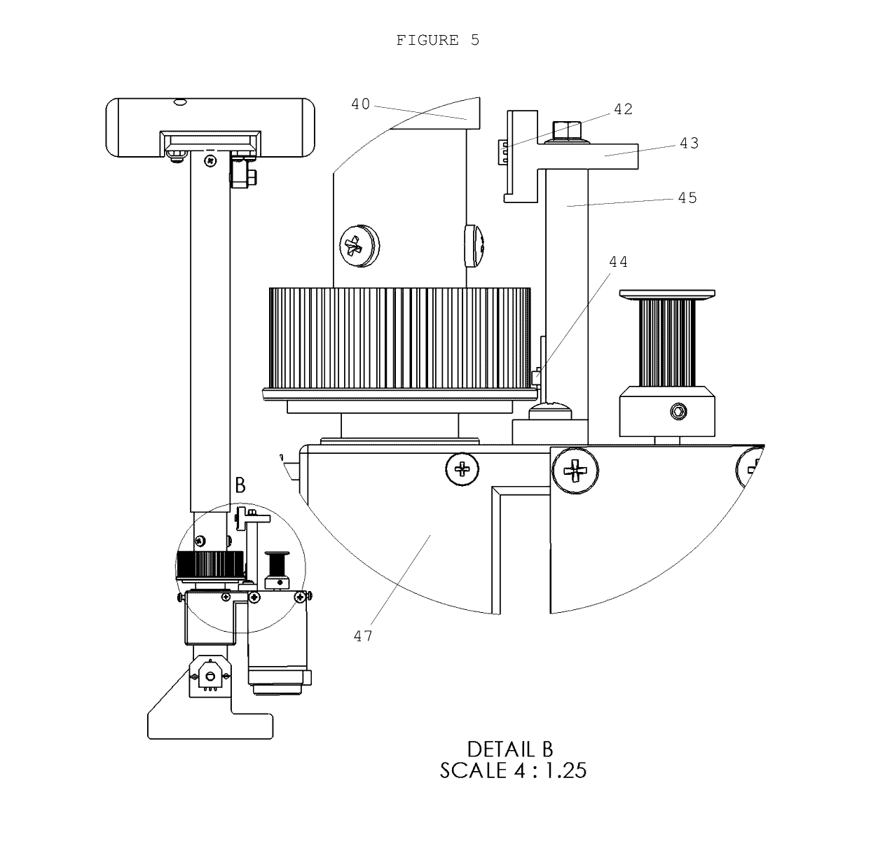

FIG. 5 is a detailed view of the embodiment for the shoulder support actuator 20 with both the actuator enclosure 30 and the pulley belt 36 removed. It shows in large scale the mounting and alignment of the retract position sensor 42 and the rotation sensor 44. Both the retract position sensor 42 and the rotation sensor 44 are mounted on slot-adjusted brackets.

The retract position sensor 42 is mounted on the sensor mounting bracket high 43, which is screw fastened on the top of the sensor mounting bracket low 45. By loosening the screw and sliding along a slot opening on the sensor mounting bracket high 43, the retract position sensor 42 can be set to closer or further away from the bottom edge of the pushrod shell 40, which is important to acquire a strong signal for a transition when the bottom edge of the pushrod shell 40 pass in front of the retract position sensor 42 on its way down, or on its way up. By slightly turn the sensor mounting bracket high 43 around the screw, the orientation, which is the alignment of the retract position sensor 42, can be adjusted as well. For the embodiment, a proximity sensor, a Hall Effect sensor in particular, is selected as the retract position sensor 42. A photo-sensitive proximity sensor can be just as good replacing the Hall Effect sensor in other embodiments. The retract position sensor 42 monitors and feeds back the pushrod shell 40 bottom edge passing event to the controller 26. The pushrod shell 40 bottom edge passing event is used as indicator that the shoulder support pad 28 be at its lower limit position.

The rotation sensor 44 is mounted on the sensor mounting bracket low 45, which is screw fastened on the top surface of the actuator housing 47. By loosening the two screws and sliding along the two slot openings on the sensor mounting bracket low 45, the rotation sensor 44 can be aligned properly against tooth edges on the pulley-driven 34, to generate strong and sharp transition when the pulley rotates. For the embodiment, a proximity sensor, a Hall Effect sensor in particular, is selected as the rotation sensor 44. In other embodiments, magnetic pickup (sensor) can be used to replace the Hall Effect sensor with nearly identical performance. An encoder can be used in yet another set of embodiments to achieve just as good results, replacing the Hall Effect sensor. However, encoder usually requires concentric mount on the rotating shaft, which means, mounting on an idler wheel(or gear), if gear set speed reduction is used. On an extremely simplified embodiment, the signal from motor encoder 50 can be tapped for this purpose as well, keeping track of the rotation position of the hollow shaft 56, which can be easily translated into the shoulder support pad 28 position away from its lower limit position, using the formula: Shoulder support pad 28 position=(accumulated tooth count/number of tooth on Pulley-driven 34)×Ball Screw Pitch. The retract position sensor 42 reported pushrod shell 40 bottom edge passing event is used to initialize (or reset) current shoulder support pad 28 position to zero.

Partly because of the need from the rotation sensor 44 for picking up tooth edge transition, for this particular embodiment, pulley-driven 34, pulley-driving 46, and the pulley belt 36 all have teeth on their engaging surface.

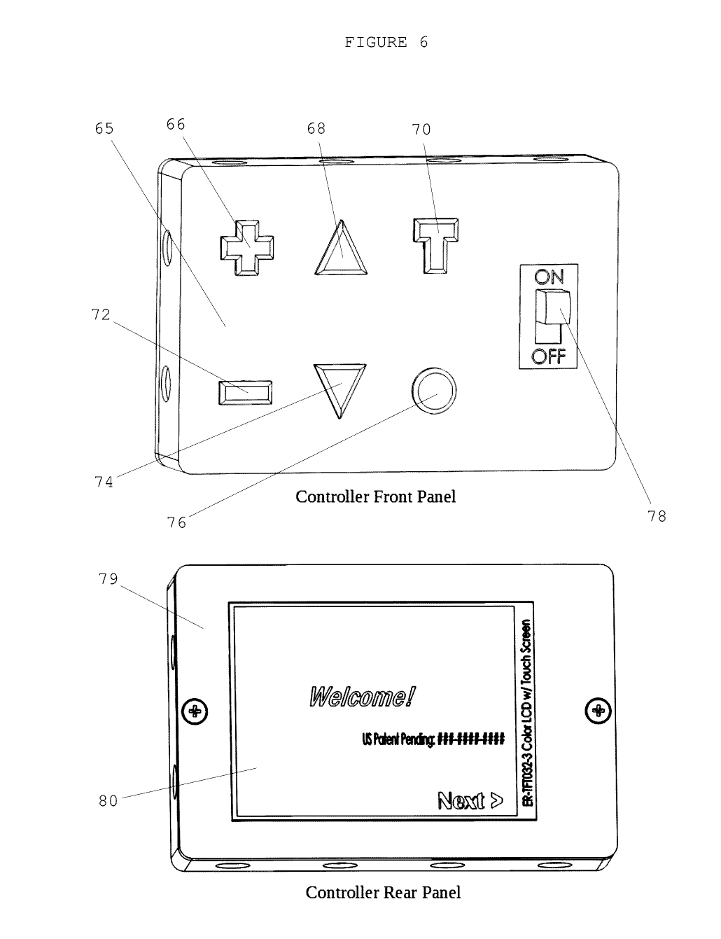

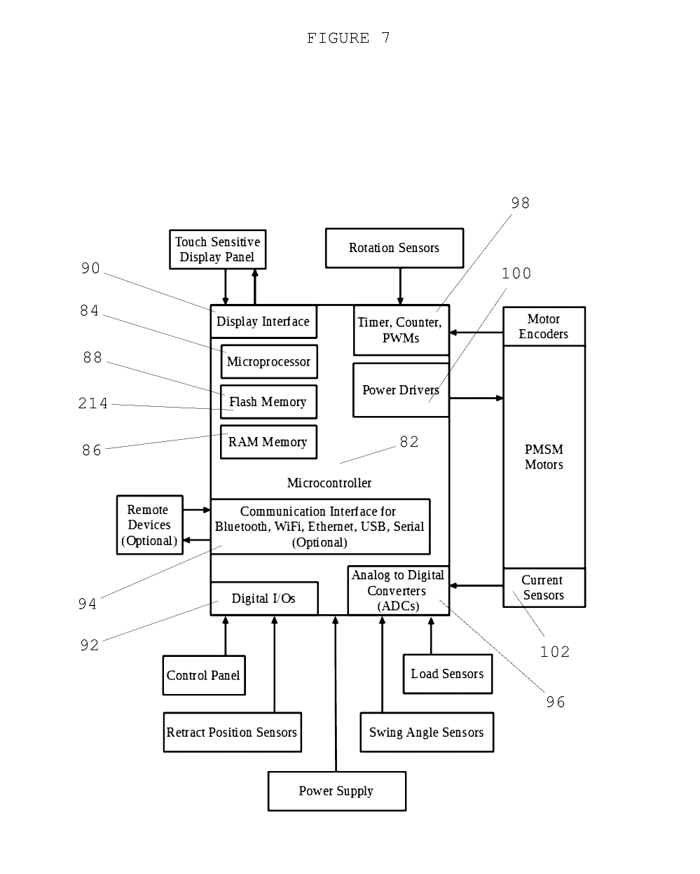

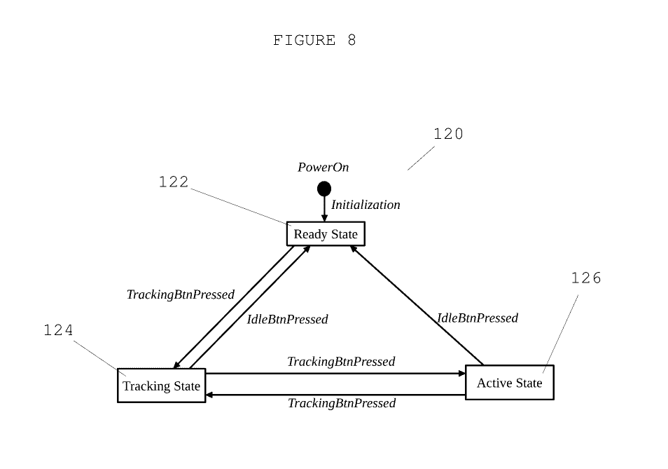

FIG. 6 is a presentation for an embodiment of the controller 26 showing its (enclosure front panel serving as) control panel 65 with power switch 78 and multiple control buttons on the top, and its rear panel 79 with a touch-sensitive display 80 on the bottom. A microcontroller-based control board (refer to FIG. 7 for more details) is enclosed in the enclosure. For this particular embodiment, there are six easy access control buttons on the control panel 65. They are Tracking button 70, Idle button 76, MoveUp button 68, MoveDown button 74, ForceUp button 66, and ForceDown button 72. The Tracking button 70 and the Idle button 76 are used to trigger state transition. MoveUp button 68 and MoveDown button 74 are only active in Ready state 122. ForceUp button 66 and ForceDown button 72 are only processed in Active state 126. Details on operations of the six control buttons will be given as part of the discussion for FIG. 8 for the state machine 120 diagram, as well as in the Details on Algorithms in Embedded Software (Firmware 214), Systems Behavior, and Operations accordingly. User control input by pressing the buttons are being processed by the microcontroller-based control board 82. Also for this particular embodiment, a touch-sensitive display 80 is integrated on the rear panel 79. Detailed configuration information can be entered using the touch-sensitive display 80. Troubleshooting is supported by using the touch-sensitive display 80. Data recording and analysis is another set of features supported on the touch-sensitive display 80. If remotely connected devices (such as smart phone, tablet, etc.) are supported, the integrated touch-sensitive display 80 becomes optional.

FIG. 7 is a diagram showing a particular embodiment of the functional blocks on the microcontroller-based control board 82 and its interfaces to the rest of the electrical and electronics components in the system. The flash memory 88 is where the embedded software (or firmware 214) and preset parameters are stored and executed from. RAM memory 86 stores all real-time data, and intermediate data. Microprocessor 84 is the program executing unit, which loads and executes instructions from the flash memory 88, responds and services to interrupt and non-interrupt I/O needs. From application point of view, the microprocessor 84 periodically executes application software, reads inputs, updates its internal states following logic and algorithms in the embedded software, drives outputs. For the particular embodiment, The main input sources are load sensors 62, rotation sensors 44, retract position sensors 42, swing angle sensors 32, motor encoders 50, current sensors 102, timers as well as user control inputs from the control buttons on control panel 65, and the touch-sensitive display 80. The outputs go to the electric motors 48. As shown in the diagram, the load sensors and the swing angle sensors are interfaced using the ADCs (Analog-to-Digit Converters). The retract position sensors and all control buttons on the control panel 65 are interfaced using digital I/Os 92. The rotation sensors are interfaced using timers. In the particular embodiment, STM32F303 by ST Microelectronics is selected as the microcontroller for the control board 82. For the touch-sensitive display 80, a particular embodiment selected an off-the-shelf part, ER-TFT-32-3 by EastRising Technology. The display interface 90 shown on the diagram is a SPI serial link enhanced with drivers complying with the vendor's API. Connecting to the control board 82 using the communication interfaces 94 (which include WiFi, Bluetooth, Ethernet, USB, Serial, etc.), remote devices, such as smart phone, tablet, or computer, can run dedicated application(s) to display operating status of the system, enter configuration, or even control inputs to interact with the system. When remote devices are available and connected, the dedicated touch-sensitive display 80 becomes optional; even the control buttons on the control panel 65 can be substituted with remote connected control buttons or virtual control buttons simulated on a touch sensitive display on remote device(s).

For detailed discussion on motor control, please refer to UM1052, User manual for STM32F PMSM single/dual FOC SDK v4.0, by ST Microelectronics, June 2014.

Details on Algorithms in Embedded Software (Firmware 214), Systems Behavior, and Operations:

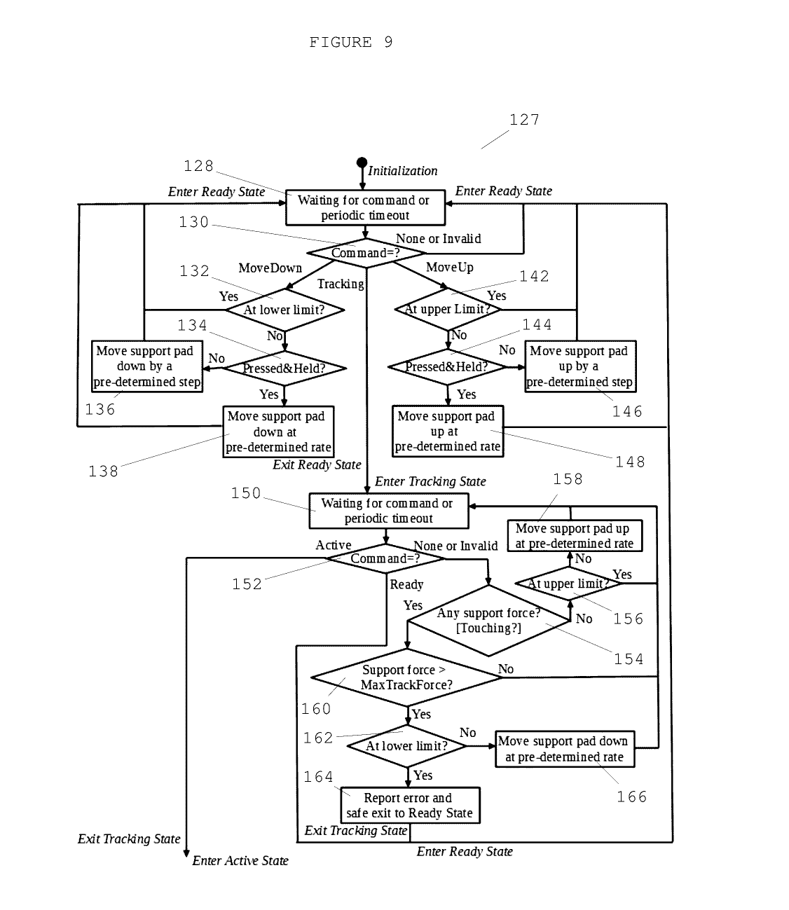

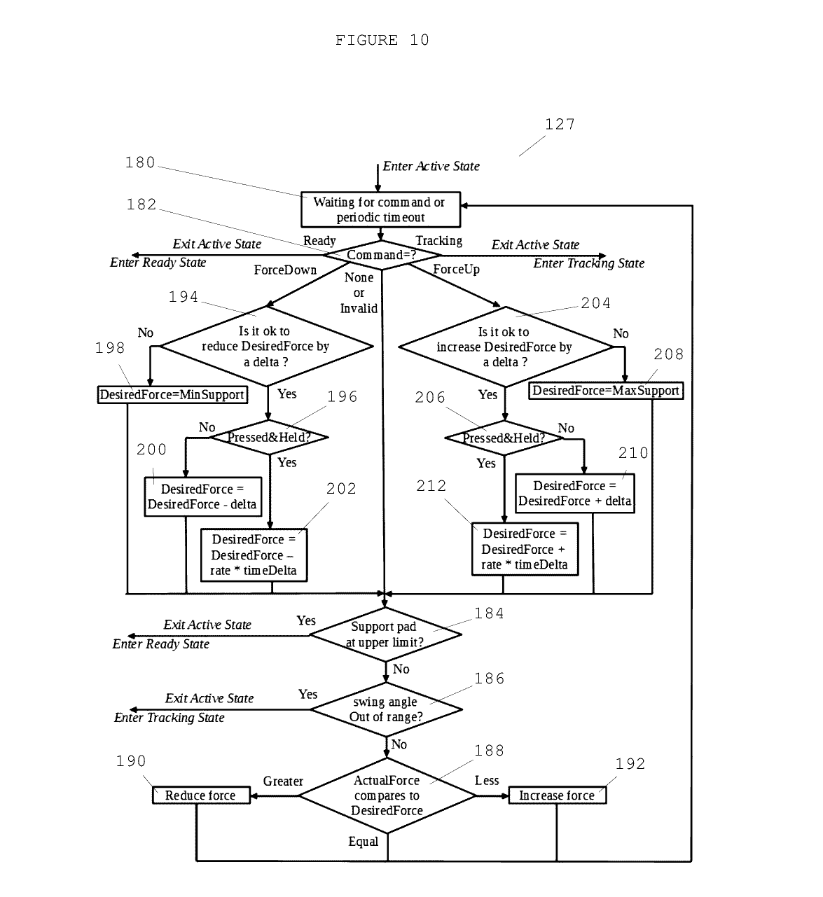

FIG. 8 is a diagram showing an embodiment of a state machine 120 to be implemented in the embedded software (firmware 214). There are three (3) states in the state machine 120. They are Ready state 122, Tracking state 124, and Active state 126. Ready state 122 is the state when the system is powered up and initialized and waiting for user input. This is the state in which the controller 26 is not driving, and it is waiting for user input. In Ready state 122, press and release the MoveUp button 68 or the MoveDown button 74, the controller 26 will drive the shoulder support pads up or down accordingly by a predetermined step; press and hold the MoveUp button 68 or the MoveDown button 74 will move the shoulder support pads 28 up or down at pre-determined rate until it either reach the bottom of shoulders or it reach the lowest positions in its up-and-down travel; press and release the Tracking button 70 will result in state transition from Ready state 122 to Tracking state 124; all other control button inputs are not responded to. Tracking state 124 is the state when the controller 26 drive the shoulder support pads 28 up until them touch the bottom of shoulder in armpits, and then, track shoulder movement with negligible support force. In Tracking state 124, press and release Idle button 76 will result in state transition from Tracking state 124 to Ready state 122; press and release the Tracking button 70 will result in state transition from Tracking state 124 to Active state 126; all other control button inputs are not responded to. Active state 126 is the state when the shoulder support pads 28 tracks the shoulder movement, and actively provide the desired upper bound shoulder support force. In Active state 126, press and release the ForceUp button 66 or the ForceDown button 72 will bump up or bump down the desired shoulder support force by a predetermined step; press and release the Idle button 76 will result in state transition from Active state 126 to Ready state 122; press and release the Tracking button 70 will result in state transition from Active state 126 to Tracking state 124; all other control button inputs are not responded to. The state machine 120 diagram is intended for capturing high level abstraction of system behavior into well defined operating states and certain norminal events that trigger state transitions. For a comprehensive description on all events (including user commands as well as limiting events and error) on state transition, please refer to the flow chart in FIG. 9 and FIG. 10.

FIG. 9 is a software flowchart showing the 1st page of an embodiment of the control algorithm to be implemented in the firmware 214 (embedded software). While the state machine 120 is in Ready state 122, it will be waiting for command or periodic timeout event. It checks for command received periodically. Upon receiving no command or invalid command; it will do nothing and simply go back to waiting for command or periodic timeout. Upon receiving Tracking command (Tracking button 70 pressed), the state machine 120 will exit Ready state 122, and enter Tracking state 124. Upon receiving MoveDown command (MoveDown button 74 pressed), it checks if the shoulder support pad 28 is already at its lower limit position. If the shoulder support pad 28 is at its lower limit position, it will do nothing, and simply go back to waiting for command or periodic timeout. If the shoulder support pad 28 is not at its lower limit position, it further checks if the MoveDown button 74 is being pressed and held down. If the MoveDown button 74 is not being pressed and held down, it will move the shoulder support pad 28 down by a pre-determined step. If the MoveDown button 74 is being pressed and held down, it will move the shoulder support pad 28 down at a pre-determined rate. Upon receiving MoveUp command (MoveUp button 68 pressed), it checks if the shoulder support pad 28 is already at its upper limit position. If the shoulder support pad 28 is at its upper limit position, it will do nothing, and simply go back to waiting for command or periodic timeout. If the shoulder support pad 28 is not at its upper limit position, it will further check if the MoveUp button 68 is being pressed and held down. If the MoveUp button 68 is not being pressed and held down, it will move the shoulder support pad up by a pre-determined step. If the MoveUp button 68 is being pressed and held down, it will move the shoulder support pad 28 up at a pre-determined rate.

While the state machine 120 is in Tracking state 124, it is waiting for command or periodic timeout event. It checks for command received periodically. Upon receiving no command or invalid command; it checks if the load sensor 62 is detecting any support force. If no support force is detected, it will further check if the shoulder support pad 28 is at its upper limit position; if the shoulder support pad 28 is at its upper limit position, it will do nothing, and simply go back to waiting for command or periodic timeout; if the shoulder support pad 28 is not at its upper limit position, it will move the shoulder support pad 28 up at pre-determined rate. If support force is detected, it will further check if the support force is greater than a preset MAXTRACKFORCE; if the support force is not greater than MAXTRACKFORCE, it will do nothing, and simply go back to waiting for command or periodic timeout. If the support force is greater than MAXTRACKFORCE, it will further check if the shoulder support pad 28 is at its lower limit position; if the shoulder support pad 28 is not at its lower limit position, it will move the shoulder support pad 28 down at pre-determined rate; if the shoulder support pad 28 is already at its lower limit position, it will report error, exit from Tracking state 124, enter Ready state 122. Upon receiving Ready command (Idle button 76 pressed), the state machine 120 will exit from Tracking state 124, enter Ready state 122. Upon receiving Active command (Tracking button 70 pressed while in Tracking state 124), the state machine 120 will exit from Tracking state 124, enter Active state 126.

FIG. 10 is the flow chart showing the 2nd page of the same embodiment of the control algorithm to be implemented in the embedded software. While the state machine 120 is in Active state 126, it is waiting for command or periodic timeout event. It checks for command received periodically. Upon receiving no command or invalid command, it further checks if the shoulder support pad 28 is at its upper limit position. If the shoulder support pad 28 is at its upper limit position, the state machine 120 exits from Active state 126, enters Ready state 122; if the shoulder support pad 28 is not at its upper limit position, it further checks if the swing angle is out of the range for providing shoulder support. If the swing angle is out of the range for providing shoulder support, the state machine 120 exits from Active state 126, enters Tracking state 124; if the swing angle is not out of the range for providing shoulder support, it compares the actual support force with the desired support force. If the actual support force is greater than the desired support force, it reduces the support force, then, it goes back to waiting for command or periodic timeout event; if the actual support force is equal to the desired support force, it does nothing, and simply goes back to waiting for command or periodic timeout event; if the actual support force is less than the desired force, it increases the support force, and then, goes back to waiting for command or periodic timeout event.

Upon receiving ForceDown command (ForceDown button 72 pressed), it checks if it is OK to reduce the desired force. If it is not OK to reduce the desired force, it sets the desired force to MinSupport; if it is OK to reduce the desired force, it further checks if the ForceDown button 72 is being pressed and held. If the ForceDown button 72 is not being pressed and held, it reduces the desired force by a pre-determined delta; if the ForceDown button 72 is being pressed and held, it reduces the desired force at a pre-determined rate. After the above operations dedicated to receiving ForceDown command, it further goes through all the operations defined and described for receiving no command or invalid command while in Active state 126.

Upon receiving ForceUp command (ForceUp button 66 pressed), it checks if it is OK to increase the desired force. If it is not OK to increase the desired force, it sets the desired force to MaxSupport; if it is OK to increase the desired force, it further checks if the ForceUp button 66 is being pressed and held. If the ForceUp button 66 is not being pressed and held, it increases the desired force by a pre-determined delta; if the ForceUp button 66 is being pressed and held, it increases the desired force at a pre-determined rate. After the above operations dedicated to receiving ForceUp command, it further goes through all the operations defined and described for receiving no command or invalid command while in Active state 126.

Upon receiving Tracking command (Tracking button 70 pressed), the state machine 120 exits Active state 126, enters Tracking state 124. Upon receiving Ready command (Idle button 76 pressed), the state machine 120 exits Active state 126, enters Ready state 122.

With the algorithms and embedded software as described in the embodiment, the following is a description of a typical operation scenario. After power On the system, as part of the initialization, shoulder support pads 28 retract (move down) until the bottom edges of the pushrod shells 40 register with the retract position sensors 42 as the shoulder support pads 28 reach their lower limit position, and the state machine 120 is at Ready state 122. While in Ready state 122, press and release the MoveUp button 68 will move the shoulder support pads 28 up by a pre-determined step, while press and hold the same button will move the shoulder support pads 28 up at pre-determined rate until reaching either the bottoms of shoulders or their upper limit position; press the MoveDown button 74 will move the shoulder support pads 28 down by a pre-determined step, while press and hold the same button will move the shoulder support pads 28 down at pre-determined rate until reaching their lower limit position. Press on the Tracking button 70, the state machine 120 exits Ready state 122, enters Tracking state 124. The two shoulder support pads 28 move up until touching shoulders in armpits. From there on, the shoulder support pads 28 provide no meaningful support force while following the up-and-down motion by the shoulders. While in Tracking state 124, press the Tracking button 70 will toggle the state machine 120 exiting Tracking state 124, entering Active state 126. Also in Tracking state 124, press the Idle button 76 will toggle the state machine 120 exiting Tracking state 124, entering Ready state 122. In Active state 126, press and release the ForceUp button 66 will cause the support force to increase by a pre-determined step, while press and hold the same button will cause the support force to increase at pre-determined rate up to maximum allowed support force; press and release the ForceDown button 72 will cause the support force to decrease by a pre-determined step, while press and hold the same button will cause the support force to decrease at pre-determined rate up to minimum allowed support force. In Active state 126, press the Tracking button 70 will toggle the state machine 120 exiting Active state 126, entering Tracking state 124. Also in Active state 126, press the Idle button 76 will cause the state machine 120 to exiting Active state 126, entering Ready state 122.

Alternative Solutions:

The shoulder support actuator 20 focused in FIG. 3, FIG. 4 and FIG. 5 is an embodiment with shoulder support pad 28 mounted on top of a linear actuator 27 using rotating electric motor 48 as power source, belt-pulley 33 mechanism for speed reduction and augmentation of torque, ballscrew 55 mechanism for rotation to linear motion conversion. Alternatively, in other embodiments, other mechanisms and their combinations can potentially be used to achieve varying, but may be acceptable results. These other mechanisms, such as but not limited to, are ballscrew 55 replacements such as threadless ball screw, rolling ring drive, lead screw, acme lead screw, rack and pinion, belt-pulley 33 replacement such as gear reduction with one or more stages of gear sets, belt-pulley 33 replacement using chain and sprockets for speed reduction. The linear actuator 27 driving the shoulder support pad 28 with rotating electric motor 48, ballscrew 55 and belt-pulley 33 combination can be replaced with linear motor direct drive, hydraulic or pneumatic powered piston driven linear actuators.

There are a variety of different embodiments possible for the control panel 65 with control buttons supporting frequently used commands. The ideal number of commands and buttons to be included on the control panel 65 are subjective to individual preference and can vary. On one extreme embodiment, the system can function with no control button at all, hence no need for a control panel 65 other than a power switch 78 or button, and relying on the power switch 78 or power button to set the system to Active state 126 when powered ON, and retract the shoulder support pad 28 to its lower limit position when powered OFF. Not a nice and comprehensive behavior, but probably will be OK with a few users. On another embodiment there can be only one control button in addition to the power switch 78 or button, toggling between Ready state 122 and Active state 126, or sequencing among Ready state 122, Tracking state 124 and Active state 126, while not handy to most people but may be acceptable to some. A third embodiment with two control buttons in addition to power switch 78 or button, one for Idle button 76 which forces enter to Ready state 122, and the second as the Tracking button 70 for entering Tracking state 124 from Ready state 122, and also for toggling between Tracking state 124 and Active state 126, would be entirely acceptable for a stripped down implementation. Embodiments with more than six control buttons will function as well, albeit confusing to some users. In yet another embodiment, the control panel 65 can be a standalone device by itself, connecting to the controller 26 (microcontroller-based control board 82) with wire, or wireless via technologies such as but not limited to Bluetooth or WiFi. Further more, there is a potential embodiment with the control panel 65 integrated with touch-sensitive display 80 forming a standalone device connecting to the controller 26 with wire, or wireless using technologies such as but not limited to Bluetooth or WiFi. In embodiments where remotely connected device(s), such as but not limited to smart phone, tablet or computer, is available and connected, by running application on such remotely connected device, all functionalities on locally connected touch-sensitive display 80 can be accomplished; even control buttons can be substituted by virtual buttons on such remotely connected device(s). Hence, in such embodiments, both dedicated control panel 65 with control buttons, and dedicated touch-sensitive display 80 are optional.

The waist belt 24 shown in FIG. 1 and FIG. 2 is an embodiment made of soft, but strong man-made material, a flat band having two connecting ends with matching Velcro hook panels attached at the two ends for tightening around the lumbar to pelvis area in the back. Dedicated pockets 25 for housing shoulder support actuators 20, controller 26, and batteries 22 are fixed to the outer surface of the waist belt 24. In other embodiments, the material can be replaced with natural materials such as but not limited to cotton, animal hide, a mixture of natural and synthetic materials such as but not limited to cotton cloth with steel enhancement or carbon fiber enhancement, synthetic materials such as but not limited to nylon and polyurethane. In some other embodiments, the Velcro hook panel attached to the two connecting ends can be replaced with a variety of belt buckle designs with or without pin, made of a variety of materials such as but not limited to metal or plastic, achieving varying, but may be acceptable performance. For yet other embodiments, on the waist belt 24 where the two ends connect can be in the back, front, side, or any an angular position as long as the changed position poses no major compromise to tightness, load carrying performance or the functionality of the pockets for mounting shoulder support actuator 20, controller 26, or battery 22. In some other embodiments, the mounting pockets can be separate pieces from the waist belt 24, attached or hang on to the waist belt 24. Mounting pocket 25 on the waist belt 24 is a generalized term for a structure with opening on its top and provide constraint on relative motion down-ward and side-ways. As long as the item in the mounting pocket 25 is sufficiently constrained from down-ward motion and side-way motion, the mounting pocket 25 does not necessarily have to have bottom or side(s).

In the embodiment shown in the figures, the shoulder support pad 28 has a short beam like top structure which contact the under part of shoulder. In other embodiments, the top structure can be custom built to not only give a perfect fit to the individual user, but may style to the individual's taste as well by using 3D printing.

The embodiment of the wearable active upper body support system 16 shown in FIG. 1 and FIG. 2 has two pieces of batteries 22. In some other embodiments, single piece of battery 22, or more than two pieces of batteries 22 are just as acceptable.

The embodiment of the active shoulder support device 18 in FIG. 1 and FIG. 2 contains two pieces of shoulder support actuators 20. Other embodiments may typically contain shoulder support actuators 20 in even number of pieces such as 2, 4, 6, or more for balanced support on both left and right shoulders. However, there may be embodiments where unbalanced support is needed which make odd number of shoulder support actuators 20 in an active shoulder support device 18 also possible, such as 1, 3, 5 or more.

### REFERENCES:

1) UM1052, User manual for STM32F PMSM single/dual FOC SDK v4.0, by ST Microelectronics, June 2014.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A wearable active upper body support system for counter gravity relief to spine, comprising:
means for providing computer controlled active shoulder support by positioning shoulder support actuators along sides of the ribcage under the left and the right shoulders in armpits;
means for providing up-and-down motion and shoulder support force from armpit under shoulder, subassembly assembled to said means for providing computer controlled active shoulder support by positioning shoulder support actuators along sides of the ribcage under the left and the right shoulders in armpits;
means for primary energy source for the wearable active upper body support system;
means for tightening around the lumbar to pelvis area for serving as the housing and anchoring base for shoulder support actuator, and optionally with mounting pockets for housing the rest of the system;
means for taking input for controls and configuration, monitoring feedback from sensors, executing firmware loaded in its memory, and manage the support force by driving power source, electrically connected to said means for primary energy source for the wearable active upper body support system, and subassembly assembled to said means for providing computer controlled active shoulder support by positioning shoulder support actuators along sides of the ribcage under the left and the right shoulders in armpits;
means for generating the desired up-and-down linear motion and the support force, subassembly assembled to said means for providing up-and-down motion and shoulder support force from armpit under shoulder;
means for establishing firm and comfortable contact with the bottom of shoulder in armpit and conveying the upper bound shoulder support force, mounted to said means for generating the desired up-and-down linear motion and the support force, and subassembly assembled to said means for providing up-and-down motion and shoulder support force from armpit under shoulder;
means for feeding back to controller in real-time the actual support force exerted on the shoulder support pad, electrically connected to said means for taking input for controls and configuration, monitoring feedback from sensors, executing firmware loaded in its memory, and manage the support force by driving power source, and subassembly assembled to said means for providing up-and-down motion and shoulder support force from armpit under shoulder; and
means for implementing the desired algorithms in microcontroller executable code.

2. The wearable active upper body support system in accordance with claim 1, wherein said means for providing computer controlled active shoulder support by positioning shoulder support actuators along sides of the ribcage under the left and the right shoulders in armpits comprises an active shoulder support device.

3. The wearable active upper body support system in accordance with claim 1, wherein said means for providing up-and-down motion and shoulder support force from armpit under shoulder comprises a shoulder support actuator.

4. The wearable active upper body support system in accordance with claim 1, wherein said means for primary energy source for the wearable active upper body support system comprises a battery.

5. The wearable active upper body support system in accordance with claim 1, wherein said means for tightening around the lumbar to pelvis area for serving as the housing and anchoring base for shoulder support actuator, and optionally with mounting pockets for housing the rest of the system comprises a waist belt.

6. The wearable active upper body support system in accordance with claim 1, wherein said means for taking input for controls and configuration, monitoring feedback from sensors, executing firmware loaded in its memory, and manage the support force by driving power source comprises a controller.

7. The wearable active upper body support system in accordance with claim 1, wherein said means for generating the desired up-and-down linear motion and the support force comprises a linear actuator.

8. The wearable active upper body support system in accordance with claim 1, wherein said means for establishing firm and comfortable contact with the bottom of shoulder in armpit and conveying the upper bound shoulder support force comprises a shoulder support pad.

9. The wearable active upper body support system in accordance with claim 1, wherein said means for feeding back to controller in real-time the actual support force exerted on the shoulder support pad comprises a load sensor.

10. The wearable active upper body support system in accordance with claim 1, wherein said means for implementing the desired algorithms in microcontroller executable code comprises a firmware.

11. A wearable active upper body support system for counter gravity relief to spine, comprising:
an active shoulder support device, for providing computer controlled active shoulder support by positioning shoulder support actuators along sides of the ribcage under the left and the right shoulders in armpits;
a shoulder support actuator, for providing up-and-down motion and shoulder support force from armpit under shoulder, subassembly assembled to said active shoulder support device;
a battery, for primary energy source for the wearable active upper body support system;
a waist belt, for tightening around the lumbar to pelvis area for serving as the housing and anchoring base for shoulder support actuator, and optionally with mounting pockets for housing the rest of the system;
a controller, for taking input for controls and configuration, monitoring feedback from sensors, executing firmware loaded in its memory, and manage the support force by driving power source, electrically connected to said battery, and subassembly assembled to said active shoulder support device;
a linear actuator, for generating the desired up-and-down linear motion and the support force, subassembly assembled to said shoulder support actuator;
a shoulder support pad, for establishing firm and comfortable contact with the bottom of shoulder in armpit and conveying the upper bound shoulder support force, mounted to said linear actuator, and subassembly assembled to said shoulder support actuator;
a load sensor, for feeding back to controller in real-time the actual support force exerted on the shoulder support pad, electrically connected to said controller, and subassembly assembled to said shoulder support actuator; and
a firmware, for implementing the desired algorithms in microcontroller executable code.

12. A wearable active upper body support system for counter gravity relief to spine, comprising:
an active shoulder support device, for providing computer controlled active shoulder support by positioning shoulder support actuators along sides of the ribcage under the left and the right shoulders in armpits;
a shoulder support actuator, for providing up-and-down motion and shoulder support force from armpit under shoulder, subassembly assembled to said active shoulder support device;
a battery, for primary energy source for the wearable active upper body support system;
a waist belt, for tightening around the lumbar to pelvis area for serving as the housing and anchoring base for shoulder support actuator, and optionally with mounting pockets for housing the rest of the system;
a controller, for taking input for controls and configuration, monitoring feedback from sensors, executing firmware loaded in its memory, and manage the support force by driving power source, electrically connected to said battery, and subassembly assembled to said active shoulder support device;
a linear actuator, for generating the desired up-and-down linear motion and the support force, subassembly assembled to said shoulder support actuator;
a shoulder support pad, for establishing firm and comfortable contact with the bottom of shoulder in armpit and conveying the upper bound shoulder support force, mounted to said linear actuator, and subassembly assembled to said shoulder support actuator;
a swing angle sensor, for feeding back the support pad swing angle to controller for swing angle range related control features, electrically connected to said controller, and subassembly assembled to said shoulder support actuator;
a belt-pulley, for reducing the rotational speed from electric motor, and augmenting its torque, subassembly assembled to said linear actuator;
a retract position sensor, for feeding back pushrod shell bottom edge passing event to controller for indicating shoulder support pad at its lower limit position in travel, electrically connected to said controller, and subassembly assembled to said shoulder support actuator;
a rotation sensor, for feeding back real-time rotation status to controller for determining shoulder support pad travel distance away from its lower limit position, electrically connected to said controller, and subassembly assembled to said shoulder support actuator;
an electric motor, for power source, subassembly assembled to said linear actuator, and electrically connected to said controller;
a ballscrew, for converting rotation to linear motion, subassembly assembled to said linear actuator;
a load sensor, for feeding back to controller in real-time the actual support force exerted on the shoulder support pad, electrically connected to said controller, and subassembly assembled to said shoulder support actuator;
a torque limiter, for limiting the maximum torque allowed to be carried between the linear actuator and the shoulder support pad;
a control panel, for easy access to control buttons and power switch, subassembly assembled to said controller;
a touch-sensitive display, for detailed configuration and troubleshooting; and
a firmware, for implementing the desired algorithms in microcontroller executable code.
